(19) United States
(12) Patent Application Publication
(10) Pub. No.: US 2015/0272222 A1
(43) Pub. Date: Oct. 1, 2015

(54) INHALATION SENSOR FOR ALTERNATIVE NICOTINE/THC DELIVERY DEVICE

(71) Applicant: Nicotech, LLC, New York, NY (US)

(72) Inventors: Kristofer Spinka, New York, NY (US); Xabay Spinka, New York, NY (US)

(21) Appl. No.: 14/533,874

(22) Filed: Nov. 5, 2014

Related U.S. Application Data
(63) Continuation-in-part of application No. 14/483,828, filed on Sep. 11, 2014.
(60) Provisional application No. 61/970,238, filed on Mar. 25, 2014.

(51) Int. Cl. A24F 47/00; G01N 33/94

(52) U.S. Cl. CPC A24F 47/008; G01N 33/94

(57) ABSTRACT

An inhalation sensor for an alternative nicotine or THC delivery device having a diluted nicotine or THC solution. The inhalation sensor includes a processor and memory. The memory includes a solution profile including information indicative of the nicotine or THC concentration of the diluted nicotine or THC solution and a device profile including information indicative of the alternative nicotine or THC delivery device. The inhalation sensor is configured to determine an amount of nicotine or THC consumed by a user of the alternative nicotine or THC delivery device based on the solution profile and the device profile.

### CROSS REFERENCE TO RELATED APPLICATION

This application claims priority to U.S. application Ser. No. 14/483,828, filed Sep. 11, 2014, and U.S. Provisional Application No. 61/970,238, filed Mar. 25, 2014, the entire contents of which is incorporated herein by reference.

### BACKGROUND

1. Field of the invention

This disclosure relates to an inhalation sensor configured to determine and/or regulate an amount of nicotine and/or tetrahydrocannabinol (THC) delivered by the alternative nicotine/THC delivery device.

2. Background of the Invention

Alternative nicotine delivery devices (such as electronic cigarettes, vaporizers, and tobacco furnaces) are a widely popular means of nicotine delivery. Because they are more analogous to traditional cigarettes than other nicotine delivery devices like gums or patches, it is easier for most users to transition from traditional cigarettes to alternative nicotine delivery devices.

Traditional cigarettes, by way of the smoke itself, create a self-limiting maximum rate of nicotine consumption because inhaling traditional cigarette smoke at an increased rate creates levels of discomfort (for example, coughing, carbon monoxide, heat from the smoke, etc.). The alternative nicotine delivery device, however, poses a risk of increased nicotine consumption because the almost-sensationless effect of inhaling the vaporized nicotine solution does not have the self-limiting side effects of traditional cigarettes. Due to inconsistency in manufacturing, the amount of nicotine delivered by each alternative nicotine delivery device may vary from unit to unit. Therefore, nicotine consumption per alternative nicotine delivery device cannot be reliably tracked.

Inhalation of THC is being decriminalized and/or legalized in an increasing number of jurisdictions. Similar to nicotine, THC may be inhaled by an alternative delivery device which does not produce the self-limiting side effects of traditional cigarette.

### SUMMARY

One embodiment of the present invention is an inhalation sensor for an alternative nicotine/THC delivery device having a diluted nicotine/THC solution. The inhalation sensor includes a processor and memory. The memory includes a solution profile including information indicative of the nicotine/THC concentration of the diluted nicotine/THC solution and a device profile including information indicative of the alternative nicotine/THC delivery device. The inhalation sensor is configured to determine an amount of nicotine/THC consumed by a user of the alternative nicotine/THC delivery device based on the solution profile and the device profile.

In some instances, the simple act of informing the user regarding nicotine/THC consumption may reduce the nicotine/THC consumption of the user. In other instances, the user may want to be restricted from exceeding a predetermined maximum nicotine/THC consumption level. Accordingly, the alternative nicotine/THC delivery device may restrict the amount of nicotine/THC consumed by the user based on a predetermined nicotine/THC consumption amount or user input.

Exemplary embodiments of the present invention may enable an alternative nicotine delivery device to be classified as a smoking cessation device rather than a simple nicotine delivery system. This classification may enable the alternative nicotine delivery device to be purchased through health insurance providers and/or avoid the burdens of additional regulation.

### BRIEF DESCRIPTION OF THE DRAWINGS

Exemplary embodiments will be set forth with reference to the drawings, in which:

FIG. 1A illustrates an alternative nicotine/THC delivery device according to an exemplary embodiment of the present invention;

FIG. 1B illustrates an alternative nicotine/THC delivery device according to another exemplary embodiment of the present invention;

FIG. 1C illustrates an alternative nicotine/THC delivery device according to another exemplary embodiment of the present invention;

FIG. 2 illustrates an inhalation sensor according to an exemplary embodiment of the present invention;

FIG. 3 illustrates an alternative nicotine/THC delivery device according to another exemplary embodiment of the present invention;

FIG. 4 illustrates an alternative nicotine/THC delivery device according to yet another exemplary embodiment of the present invention;

FIG. 5 illustrates an alternative nicotine/THC delivery device according to yet another exemplary embodiment of the present invention;

FIG. 6 illustrates an alternative nicotine/THC delivery device according to yet another exemplary embodiment of the present invention; and

FIG. 7 illustrates another view of the charging unit illustrated in FIG. 1B and/or the charging unit illustrated in FIG. 1C according to an exemplary embodiment of the present invention.

### DETAILED DESCRIPTION OF EXEMPLARY EMBODIMENTS

Exemplary embodiments of the present invention will be set forth in detail with reference to the accompanying drawings, in which like reference numerals refer to like elements throughout. The description set forth below and illustrated in part by the drawings is intended to serve as a description of exemplary embodiments of the application and is not intended to represent the only methods by which the present application can be constructed and/or utilized. The description sets forth the functions and the sequence of steps for constructing, calibrating and operating exemplary embodiments of the present invention. It is to be understood, however, that the same or equivalent functions and sequences can be accomplished by different embodiments that are also intended to be encompassed within the spirit and scope of this disclosure. Exemplary embodiments illustrated in the accompanying drawings are not necessarily to scale and are instead provided to convey the inventive concepts to one of ordinary skill in the art.

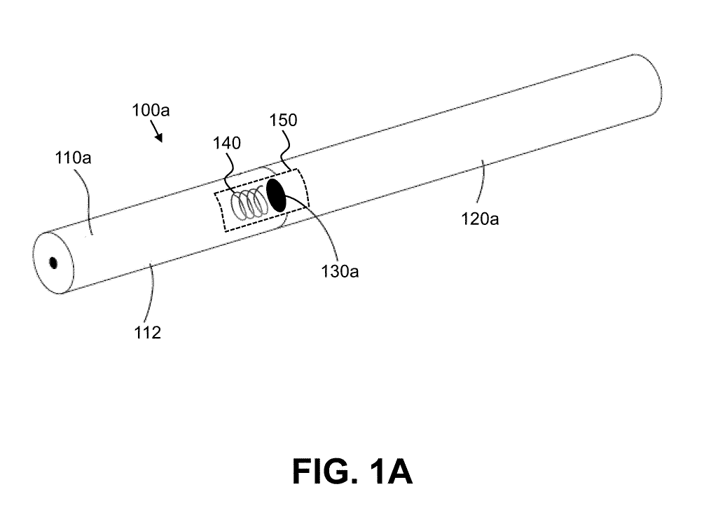

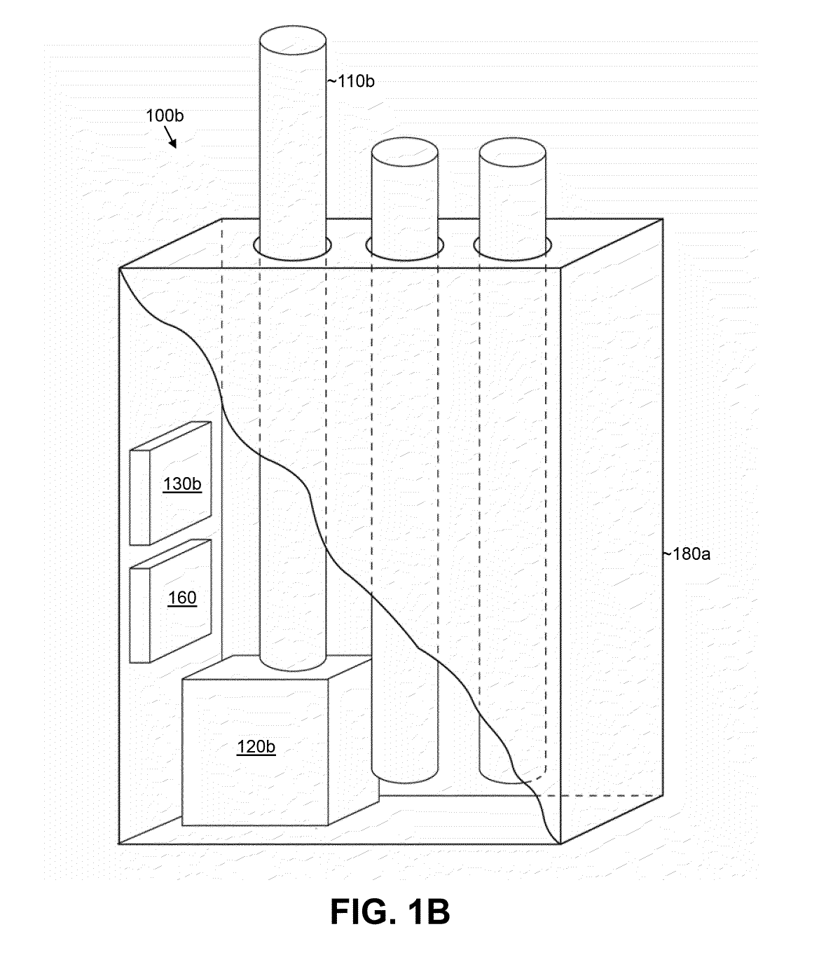

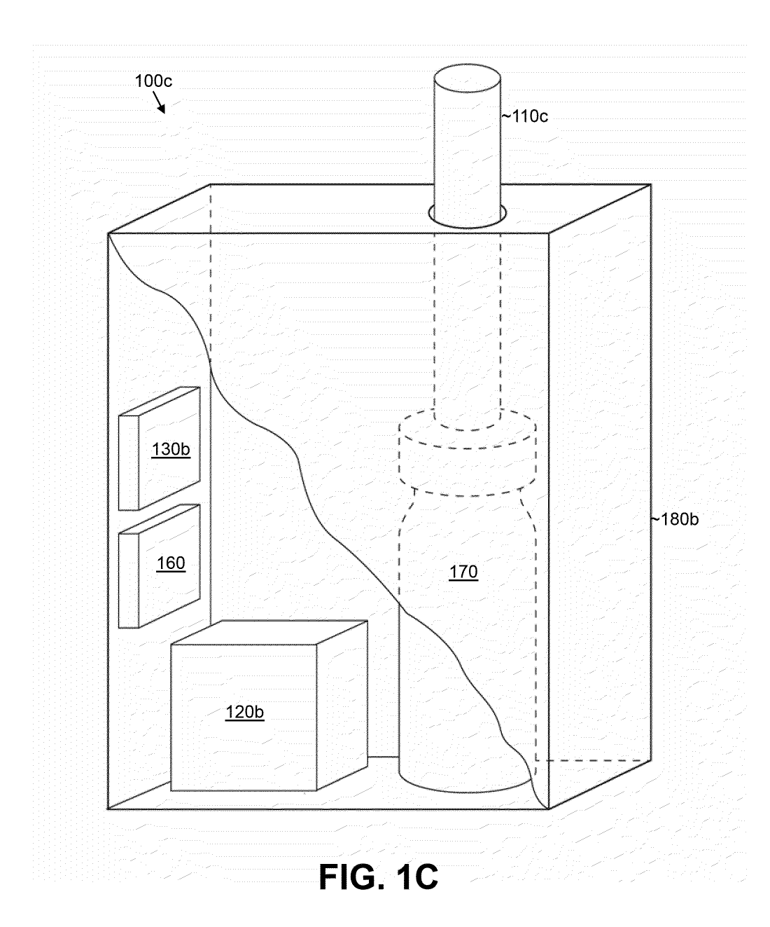

FIG. 1A illustrates an alternative nicotine/THC delivery device 100a, including a reservoir 110a for storing a diluted nicotine/THC solution 112, a power source 120a, electronic circuitry 130a, and a heating element 140 (shown inside cut-out 150). The device 100a may also include a negative pressure switch or manually actuated switch (not shown). The electronic circuitry 130a may include a processor and may be configured to electrically connect the power source 120a and the heating element 140 to vaporize the solution 112 in response to the switch. The electronic circuitry 130a may also provide very basic functions including passing current to a decorative light emitting diode (LED), regulating electrical current flow to the heating element 140, and limiting the contiguous amount of time the heating element 140 can be in use for one inhalation (i.e., draw, usage, puff) of the device in order to protect the solution 112 and heating element 140 from overheating and releasing toxic substances from the nicotine/THC solution (e.g., formaldehyde).

While the device 100a may be an electronic cigarette including the reservoir 110a for storing a diluted nicotine/THC solution 112, in another exemplary embodiment the device 100a may be any other vaporizing device configured to deliver any vaporized solution (with or without nicotine or THC). In yet another exemplary embodiment, the 100 may be a tobacco furnace including a nicotine/THC cartridge (for example, a cartridge containing tobacco or cannabis and a filter) in addition to or instead of the reservoir 110a for storing the solution 112.

The reservoir 110a may be refillable or disposable. The power source 120a may be a battery, a fuel injector, or any other device configured to supply power to the heating element 140. The power source 120a may be rechargeable or disposable. The power source 120a may be configured to recharge via a wired or wireless connection, by mating with a charging unit as discussed below, and/or by harvesting power from sources such as radio waves, heat, light, motion, etc. The reservoir 110 may be removably connected to the power source 120a or the reservoir 110a and the power source 120a may be integrated into a single device. The electronic circuitry 130a may be incorporated as part of the power source 120a, the reservoir 110a or as a separate, removably connectable device.

FIG. 1B illustrates an alternative nicotine/THC delivery device 100b according to another exemplary embodiment of the present invention. The device 100b may include one or more inhalation units 110b and a charging unit 180a. Similar to the device 100a, the inhalation units 110b may include a reservoir for storing a diluted nicotine/THC solution 112, a power source, and a heating element to vaporize the solution 112.

The charging unit 180a may include a power source 120b, electronic circuitry 130b, and memory 160. The power source 120b may be configured to store and transfer power to the power source of the inhalation units 110b. For example, the power source of the inhalation unit 110b may be a rechargeable battery and the power source 120b (which may also be a rechargeable battery) may store electric power and charge and recharge the battery of one or more of the inhalation units 110b when one or more of the inhalation units 110b is coupled with the charging unit 180a.

The memory 160 may be any suitable device configured to store data. For example, the memory 160 may be non-volatile memory. The electronic circuitry 130b may include a processor (similar to the electronic circuitry 130a).

FIG. 1C illustrates an alternative nicotine/THC delivery device 100c according to another exemplary embodiment of the present invention. The device 100c may include a compressed air inhalation unit 110c and a charging unit 180b. Similar to the charging unit 180a, the charging unit 180b may include a power source 120b, electronic circuitry 130b, and memory 160.

The charging unit 180b may include a compressed nicotine/THC vapor reservoir 170 configured to store the nicotine/THC solution 112 under pressure. Unlike the inhalation unit 110b, the inhalation unit 110c may not include a power source or a heating element. Instead, the device 100c may be configured such that the reservoir 170 fills and/or refills the compressed air inhalation units 110c with a pressurized nicotine/THC solution 112 when the compressed air inhalation unit 110c mates with the charging unit 180b. In this instance, the compressed air inhalation unit 110c is configured to deliver the pressurized solution 112 to the user in response to an inhalation.

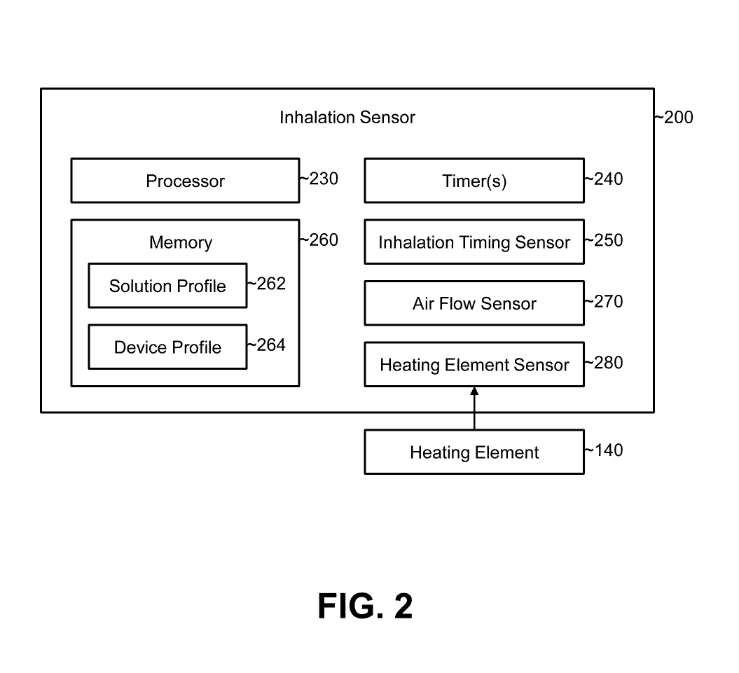

FIG. 2 illustrates an inhalation sensor 200 according to an exemplary embodiment of the present invention. The inhalation sensor 200 includes a processor 230, at least one timer 240, and memory 260. The memory 260 may include a solution profile 262 and a device profile 264. The inhalation sensor 200 may also include an inhalation timing sensor 250, an air flow sensor 270, and/or a heating element sensor 280.

The processor 230 may be an integrated circuit or soft logic processor. The memory 260 may be any non-transitory computer-readable storage medium, such flash memory, configured to store data and instructions that, when executed by the processor 230, carry out relevant portions of the features described herein.

The timer(s) 240 may be any device configured to measure time intervals. Each of the one or more timers 240 may be “always on” (real-time clock) and measure time intervals as long as the device 100a-c is connected to a power source or the device 100a-c may include a switch to connect and disconnect power to one or all of the timers 240. In either instance, the timer or timers 240 may be configured to measure time intervals both during and after an inhalation of the device 100a-c.

The inhalation timing sensor 250 may be any device configured to determine if a user is actively inhaling the solution 112. The inhalation timing sensor 250 may be configured to detect, for example, the output of the negative pressure switch described above, negative pressure from the user, air flow, flow of the solution 112 from the reservoir 110a, etc. The air flow may be determined by an optional air flow sensor 270 (discussed below).

As described in more detail below, the inhalation sensor 200 may be incorporated within the device 100a, the inhalation unit 110b, the charging unit 180, an external device, or any combination thereof. In one example, the inhalation sensor 200 may be incorporated within the device 100a (for example, as part of the electronic circuitry 130a).

In another example, the inhalation sensor 200 may be incorporated within the device 100b. In this example, the memory 160 may include the memory 260; the electronic circuitry 130b may include the processor 230 and a timer 240; and the inhalation unit 110b may include a timer 240, the inhalation timing sensor 250, the air flow sensor 270 and/or the heating element sensor 280. In this example, the electronic circuitry 130b and the inhalation unit 110b may include communications circuitry configured to send and receive data between the electronic circuitry 130b and the inhalation unit 110b. The communications between the electronic circuitry 130b and the inhalation unit 110b may be via wired connection configured to transfer data when the inhalation unit 110b is paired with the charging unit 180 or wireless connection (e.g., Bluetooth, near field communication, etc.)

In another example, the inhalation sensor 200 may be wholly or partially incorporated within an external device (e.g., a computer, a smart phone, a smart watch, a fitness tracker, etc.). In this example, the external device may include the processor 230, the memory 260, the timer 240; and the device 100a-c may include the inhalation timing sensor, the air flow sensor, and/or the heating element sensor. The external device and the device 100a-c may include wired or wireless communications circuitry as described above.

The inhalation sensor 200 is configured to determine (i.e., estimate and/or measure) the amount of nicotine or THC consumed by a user. One factor used to determine the amount of nicotine or THC consumed by a user is the nicotine/THC content of the solution 112. The nicotine/THC content of the solution 112 may be pre-determined (for example, by analyzing the solution 112 with a spectrometer tank) and stored in the memory 260 of as part of the solution profile 262. The solution profile 262 may also include additional information regarding the solution 112. For example, the solution profile 262 may include the burning point of the solution 112 (i.e., the temperature at which the solution 112 begins releasing toxic substances to the user).

In one exemplary embodiment, the device 100a-c may be configured to pair with a single diluted nicotine/THC solution 112 (e.g., a single use electronic cigarette, an electronic cigarette with one type of mechanically compatible reservoir 110a pre-filled with a single type of solution 112, etc.). In this embodiment, the solution profile 264 associated with the solution 112 may be pre-stored in the memory 260 of the inhalation sensor 200.

In another exemplary embodiment, the device 100a-c may be configured to pair with a multiple diluted nicotine/THC solutions 112 (e.g., an electronic cigarette with multiple compatible reservoirs 110a, a refillable vaporizer, etc.). In this embodiment, the solution profile 262 may be selected by the user based on the solution 112 paired with the device 100a-c. The solution profile 262 associated with the solution 112 may be downloaded from the internet via an external device (e.g., a computer, a smart phone, etc.) and transferred to the inhalation sensor 200 via a wired (e.g., USB) or wireless connection (e.g., Bluetooth). The solution profile 262 associated with the solution 112 may be determined and/or distributed by the manufacturer of the solution 112 or a third party (for example, NicoTech, LLC).

The amount of nicotine or THC consumed by a user may be further based on the type of solution 112 (e.g., a vegetable glycerin solution, a propylene glycol solution, etc.). Accordingly, the solution profile 262 may include information indicative of the solution type and the inhalation sensor 200 may further determine the amount of nicotine or THC consumed by the user based on the solution type.

In instances where the device 100a-b includes a heating element 140, another factor used to determine the amount of nicotine or THC consumed by a user may be the temperature of the heating element 140. After the heating element 140 is activated during inhalation, the temperature of the heating element 140 rises. Accordingly, the temperature of the heating element 140 is dependent on the length of an inhalation. Accordingly, the inhalation sensor 200 may be configured to determine information indicative of the temperature of the heating element 140 based on the length of an inhalation.

The inhalation sensor 200 may determine the length of each inhalation based on the output of the inhalation timing sensor 250 and the timer 240 which may be incorporated, for example, in the device 100a or the inhalation unit 112b. For example, the inhalation timing sensor 250 may output signals indicative of the beginning and end of an inhalation and the inhalation sensor 200 may determine the elapsed time between the beginning and the end of the inhalation based on the output of the timer 240.

The relationship between the length of an inhalation and the temperature of the heating element 140 may be pre-determined (for example, by simulating the use of a prototypical alternative nicotine/THC delivery device and measuring the physical characteristics of the device) and stored in the memory 260 as part of the device profile 264. The device profile 264 may be determined, for example, by placing the a prototypical alternative nicotine/THC delivery device (e.g., the same model as the alternative nicotine/THC delivery device 100a-b that includes the inhalation sensor 200) in a simulated puffing device, activating the prototypical alternative nicotine/THC delivery device for a series of successive durations and measuring the temperature of the heating element 140. The device profile 264 may also include addition information regarding the device 100a-c. The additional information may be pre-determined using the simulated puffing device in combination with the spectrometer, a gas chromatograph, a volume measurement setup, etc. For example, the device profile 264 may determine if and when the device 100a-b reaches the burning point of the solution 112.

The temperature of the heating element 140 may also be dependent on the time elapsed between each inhalation. As described above, some devices limit the length of a single inhalation in order to prevent the heating element 140 from overheating and burning the solution 112. A user, however, may initiate multiple inhalations in quick succession, activating the heating element 140 before it has cooled down after the initial inhalation. Therefore, the amount of nicotine or THC consumed by the user may be further based on the time between inhalations. Accordingly, the inhalation sensor 200 may be further configured to determine the amount of nicotine or THC consumed by the user further based on the time between inhalations. Similar to the duration of each inhalation, the time between inhalations may be determined based on the output of the timer 240 and the inhalation timing sensor 250.

The inhalation sensor 200 may determine the information indicative of the temperature of the heating element 140 based on the duration of each inhalation, the time between inhalations, and information included in the device profile 254. For example, the device profile 264 may include a ramp up profile indicative of the temperature of the heating element 140 during ramp up (i.e., the temperature increase of the heating element 140 during inhalation) and a decaying profile indicative of the temperature of the heating element 140 during ramp down (i.e., the temperature decrease of the heating element 140 after the heating element is de-activated). Accordingly, the inhalation sensor 200 may be configured to determine information indicative of the temperature of the heating element 140 during a single inhalation or multiple successive inhalations based on the duration of each inhalation and the time elapsed between inhalations.

Instead of relying solely on the device profile 264, the inhalation sensor 200 may include an optional heating element sensor 280 incorporated in the device 100a or the inhalation unit 110b. The heating element sensor 280 may be configured to determine the temperature of the heating element 140, for example, based on the in-line resistance and voltage of the heating element 140 detected by the heating element sensor 280.

The amount of nicotine or THC consumed by a user may also depend on the air flow rate of the device 100a or inhalation unit 100b during each inhalation. The inhalation sensor 200 may estimate the air flow during each inhalation based on information included in the device profile 264. For example, an estimated air flow rate may be pre-determined by simulating the use of a prototypical device as described above and measuring the air flow of the prototypical device during one or more simulated inhalations. In this example, the inhalation sensor 200 may estimate the air flow during each inhalation, for example, by multiplying the estimated air flow rate by the duration of each inhalation. Alternatively, the inhalation sensor 200 may include an optional air flow sensor 270. The air flow sensor 270 may be incorporated within the device 100a or the inhalation unit 110b and may be configured to determine the air flow or air flow rate of the device 100a-b during each inhalation. The air flow sensor 270 may be any device configured to measure or estimate the air flow or air flow rate of the device 100a or the inhalation unit 100b, including a pressure gauge, a vacuum gauge, a diaphragm, an impeller setup, etc.

As described above, continuing to inhale from the device 100a-b after the temperature of the heating element 140 has reached the burning point of the solution 112 may cause the user to ingest potentially toxic substances (e.g., formaldehyde) from the solution 112. Conventional alternative nicotine/THC delivery devices may attempt to prevent the burning of a nicotine/THC solution by limiting the duration of a single inhalation. A user, however, may further increase the temperature of a heating element of a conventional alternative nicotine/THC delivery device by initiating multiple successive inhalations. As also described above, the inhalation sensor 200 of the present invention may be configured to determine the temperature of the heating element 140 and the solution profile 262 may include information indicative of the boiling point of the solution 112. Therefore, the inhalation sensor 200 may be further configured to prevent the heating element 140 from boiling the solution 112 (even when the user initiates successive inhalations) by comparing the temperature of the heating element 140 (as determined by the inhalation sensor 200) and the boiling point of the solution 112 and outputting a signal if the temperature of the heating element 140 is approaching or exceeds the boiling point of the solution 112. The device 100a-b may be configured to prevent the heating element 140 from approaching or exceeding the boiling point of the solution 112 (e.g., by disconnecting the heating element 140 from the power source 120) and/or output an audible or visual warning (e.g., via an LED or speaker) to the user if the temperature of the heating element 140 is approaching or exceeds the boiling point of the solution 112.

The inhalation sensor 200 may be further configured to determine (i.e., estimate or measure) the nicotine, cotinine, or THC levels of a user. Because nicotine and THC are metabolized primarily by liver enzymes, the nicotine, cotinine, or THC levels of a user may be further based on the liver performance of the user. Accordingly, the inhalation sensor 200 may be further configured to estimate the liver performance of the user based on static and/or dynamic biometrics of the user stored, for example, in the memory 260. The static biometrics of the user may include the body weight of the user (which may be used as an estimate of liver mass) and/or the sex, age, height, weight, and/or body type of the user (which may be used as an estimate of liver performance). The static biometrics of the user may also include the user's average water/fluid intake, sampled cotinine levels, sampled hormone levels, etc. The static biometrics of the user may be input into the inhalation sensor 200 directly or input into an external device (e.g., a computer, a smart phone, etc.), transferred to the inhalation sensor 200 via a wired or wireless connection.

The dynamic biometrics of the user may include the metabolic rate of the user. The user's metabolic rate may be determined by an external device (e.g., a fitness tracker, a fitness watch, etc.) and transferred to and stored by the inhalation sensor 200 as described above. The dynamic biometrics of the user may also include hydration of the user. The user's hydration may be similarly determined by an external device and transferred to the inhalation sensor 200. Alternatively, the device 100a -c may include a hydration sensor. For example, the hydration sensor may be located on the exterior surface of the device 100a or inhalation unit 110b-c and may determine the bioelectrical impedance of the user based on contact with the user's fingers or mouth. In this instance, the inhalation sensor 200 may estimate the user's hydration based on the bioelectrical impedance of the user.

As described above, the inhalation sensor 200 may be integrated with the device 100a-c and may be configured to receive static and/or dynamic biometrics of the user from an external device. Alternatively, the inhalation sensor 200 may be stored and executed by an external device configured to receive the duration of each inhalation, the time between inhalations, and/or the bioelectrical impedance of the user from the device 100a-b.

The ramp up profile and the decaying profile of the device 100a-c may change over time. For example, the heating element 140 may oxidize or the output of the power supply 120a-b may change. Accordingly, the inhalation sensor 200 may be configured to update the device profile 264 to account for the changing characteristics of the device 100a-c. For example, the inhalation sensor 200 may be configured to receive measurements or estimates of the nicotine, cotinine, or THC levels of a user (e.g., from an external device as described above) and update the device profile 264 based on the measured or estimated nicotine, cotinine, or THC levels of a user in order to more accurately determine the amount of nicotine or THC consumed by a user and/or the nicotine, cotinine, or THC levels of the user.

In another low-cost exemplary embodiment of the present invention, the device 100a-b may include a capacitor and the power source 120a. The power source 120a is configured to charge the capacitor. The capacitor in turn provides power to the heating element 140 for one session. In this instance, because the amount of power stored by the capacitor and delivered to the heating element 140 is predetermined, the amount of nicotine or THC consumed by a user may be pre-determined and stored in the inhalation sensor 200. Furthermore, the circuit may be arranged such that the capacitor is trickle-charged (charged slowly) from the power source 120a at a rate inverse to the half-life of nicotine or THC. Accordingly, as the concentration of nicotine or THC in the user falls, the capacitor's charge increases and allows the user to consume more nicotine or THC only as the concentration of nicotine or THC in the user falls. This embodiment provides a low-cost alternative to a realtime clock with digital logic while limiting the rate at which a user may consume nicotine or THC over time.

In embodiments where the inhalation unit 110c does not include a heating element (for example, as shown in FIG. 1C), the inhalation sensor 200 may be realized in a device external to the inhalation unit 110c (for example, the charging unit 180b or an external device) and may be configured to determine the amount of nicotine or THC consumed by the user based on the solution profile 262, the device profile 264, and the time intervals between each instance when the inhalation unit 110c is refilled by the reservoir 170.

For example, the solution profile 262 may include the nicotine or THC concentration information as described above and the device profile 264 may include information indicative of the amount of vapor stored in the inhalation unit 110c. Each time the inhalation unit 110c is refilled by the reservoir 170, the inhalation sensor 200 may assume that the inhalation included the amount of vapor stored in the inhalation unit 110c. In this instance, the inhalation sensor 200 may estimate that the inhalation took place at some time between when the inhalation unit 110c was disconnected and reconnected with the charging unit 180b.

Alternatively, the inhalation unit 110c may contain a small power source (e.g., a battery a rapid charge capacitor, etc.) that is recharged when the inhalation unit 110c is inserted into the reservoir 170. In this instance, the inhalation unit 110c may be configured to determine the time of the inhalation and may transfer information indicative of the inhalation time to the charging unit 180b when the inhalation unit 110c mates with the charging unit 180b.

The inhalation sensor 200 may be configured to output the nicotine/THC consumption information (e.g., the amount of nicotine or THC consumed by the user and/or user nicotine, cotinine, or THC levels) to the user in the form of visual or audible notification and/or output the nicotine/THC consumption information to an external device (e.g., a computer, a smart phone, a fitness tracker, a fitness watch, etc.).

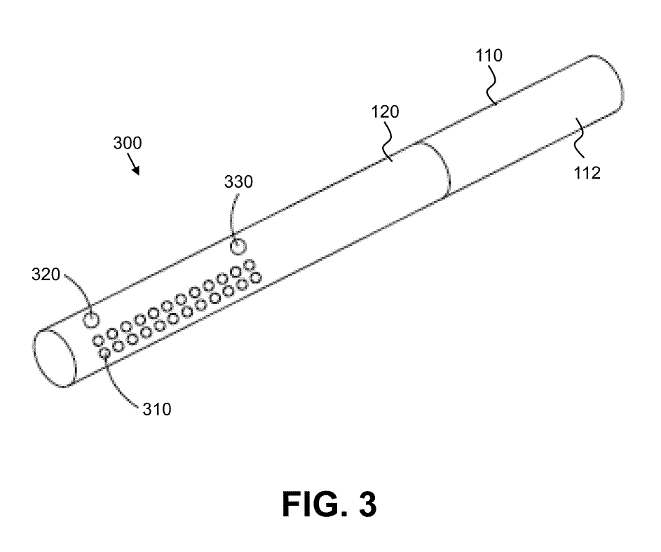

FIG. 3 illustrates an alternative nicotine/THC delivery device 300 according to an exemplary embodiment of the present invention. The device 300 may be configured to deliver the nicotine/THC solution 112 similar to the device 100a or inhalation unit 110b-c. The device 300 may also include visual indicators 310, 320, and 330 configured to output the nicotine/THC consumption information described above. As described in more detail below, the visual indicator 310, 320, and 330 may be any suitable device configured to selectively emit or reflect light. In the exemplary embodiment illustrated in FIG. 3, a plurality of visual indicators 310 emit or reflect light proportional to the nicotine or THC consumption over a predetermined time period or user nicotine, cotinine, or THC levels. The visual indicators 310 may emit or reflect light proportional to the amount of nicotine or THC consumed over the past 24 hours, the amount of nicotine or THC consumed during the current session, the estimated current plasma levels of a user, or the estimated average plasma levels of a user over the last 24 hours. The visual indicator 320 may indicate that a target minimum amount of nicotine or THC (for example, 0 mg) has been consumed over the last 24 hours. The visual indicator 330 may indicate that a predetermined maximum amount of nicotine or THC (for example, 30 mg) has been consumed over the last 24 hours.

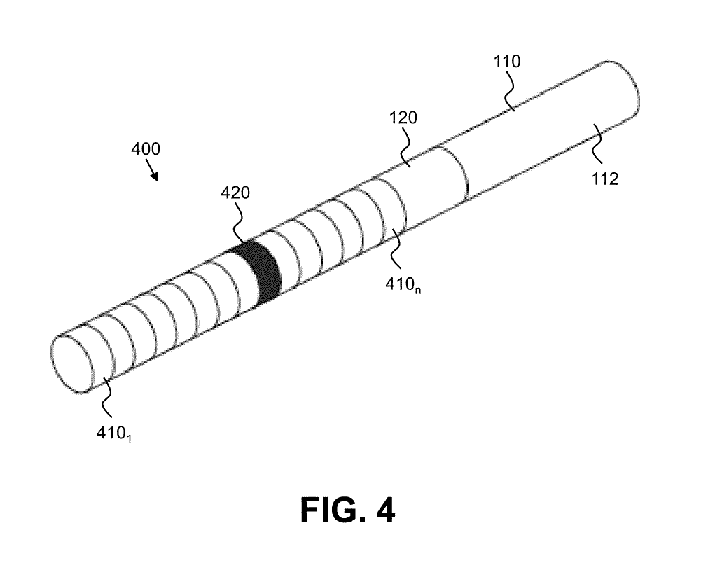

FIG. 4 illustrates an alternative nicotine/THC delivery device 400 according to another exemplary embodiment of the present invention. The device 400 may be configured to deliver the nicotine/THC solution 112 similar to the device 100a or inhalation unit 110b-c. The device 400 may also include visual indicators 410₁-410ₙ that mimic the nicotine or THC consumption (or user nicotine, cotinine, or THC levels) of a traditional cigarette. Similar to the visual indicators 310, 320, and 330, the visual indicators 410₁-410ₙ may be any suitable device configured to selectively emit or reflect light. In the exemplary embodiment illustrated in FIG. 4, the visual indicators 410₁-410ₙ emit or reflect light in succession as nicotine or THC is consumed. As shown, visual indicator 420 emits or reflects light while the other visual indicators 410₁-410ₙ are off.

The visual indicators 410₁-410ₙ may be configured to output nicotine or THC consumption information proportional to the nicotine or THC included in a traditional cigarette. For example, visual indicator 410₁ may output a visual indication at the start of a smoking session. Visual indicators 410₂-410ₙ may emit or reflect light in succession as the user continues to use the device 400. The visual indicator 410ₙ may emit or reflect light when the inhalation sensor 200 estimates that the amount of nicotine or THC consumed by the user is equivalent to the amount of nicotine or THC in a tradition cigarette.

Alternatively, the output of the visual indicators 410₁-410ₙ may be proportional to another pre-determined maximum nicotine or THC consumption or user nicotine, cotinine, or THC level. The user nicotine, cotinine, or THC levels may be based on an estimated current level and an estimated average level over a specified time period. The device 400 may audibly (e.g., via an optional speaker) or visually (e.g., via the visual indicators 410₁-410ₙ) alert the user that the nicotine or THC consumption level has reached or exceeded a predetermined maximum consumption level. Additionally or alternatively, the device 400 may audibly or visually alert the user that the nicotine or THC consumption level has fallen beneath a target threshold to notify the user when the user may resume using the device 400.

In one exemplary embodiment, the visual indicators 310, 320, 330, 410, and/or 420 may be light emitting diodes (LEDs) or any other suitable device configured to emit light. In this instance, the visual indicators 310, 320, 330, 410, and/or 420 may receive power from the power supply 120a of the device 100 or the power supply of the inhalation unit 110b.

In another exemplary embodiment, the visual indicators 310, 320, 330, 410, and/or 420 may be an electronic paper display (e.g., an electrophoretic display, an electro-wetting display, an electrofluidic display, an interferometric modulator, a micro-electro-mechanical system, etc.) or any other suitable device configured to reflect light. In this instance, the visual indicators 310, 320, 330, 410, and/or 420 may receive power from the power supply 120a of the device 100 or the power supply of the inhalation unit 110b. Alternatively, the power required to adjust the pixels of the electronic paper display may be supplied by an external device such as the charging unit 180.

In one exemplary embodiment, the visual indicators 310, 320, 330, 410, and/or 420 may be thermochromic, electrochromic, or electroluminescent polymers or paints. The polymers or paints may additionally have a color (or visibility) reversibility delay calibrated to an average of biological half-life of nicotine or THC, thus enabling the visual indicators 310, 320, 330, 410, or 420 to revert to the original color or visibility in a manner indicative of (e.g., proportional to) the nicotine, cotinine, or THC level of the user. Alternatively, micro-scale capacitors wired in series (with or without a timer) with resistors may be utilized. In this instance, information indicative of nicotine or THC consumption (or nicotine, cotinine, or THC levels) may be output by applying power to a series of emitters (e.g., LEDs) and/or activating electrochromic or electroluminescent polymers or paints and allowing the slow drain of the capacitors to restore the visual indicators to their original state in a manner indicative (e.g., proportional) of the in a manner indicative of (e.g., proportional to) the nicotine, cotinine, or THC level of the user.

In another exemplary embodiment, the visual indicators 310, 320, 330, 410, and/or 420 may be mechanical. The inhalation unit 110c of FIG. 1C, for example, may include an internal screw-based usage indicator mechanism. When the inhalation unit 110c mates with the charging unit 180b, the charging unit may rotate the screw-based usage indicator mechanism such that the visibility of colored tiles are adjusted.

The inhalation sensor 200 may limit the amount of nicotine or THC consumed over time. In instances where the device 100a-b includes a heating element 140, for example, the inhalation sensor 200 may prevent the heating element 140 from heating if the user has reached or exceeded a predetermined maximum nicotine/THC consumption level in a given time period (for example, a usage session, a daily limit, or any other measure of time) or a predetermined maximum user nicotine, cotinine, or THC level. Alternatively, the heating element 140 may be configured to output a reduced amount of heat if the user has reached or exceeded the predetermined maximum nicotine or THC consumption level in the given time period or the predetermined maximum user nicotine, cotinine, or THC level. In instances where the inhalation unit 110c does not include a heating element 140, the inhalation sensor 200 may prevent the reservoir 170 from refilling the inhalation unit 110c if the user has reached or exceeded the predetermined maximum nicotine or THC consumption level in the given time period or the predetermined maximum user nicotine, cotinine, or THC level. The predetermined maximum nicotine or THC consumption level or the user nicotine, cotinine, or THC level may be user adjustable.

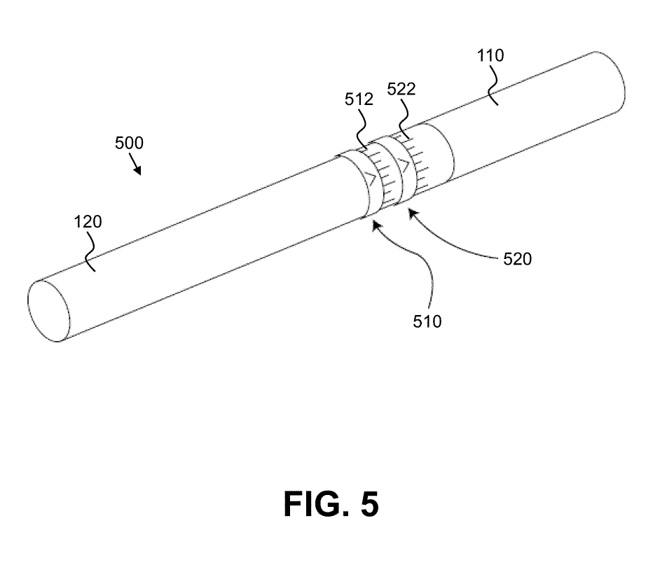

FIG. 5 illustrates an alternative nicotine/THC delivery device 500 according to another exemplary embodiment of the present invention. The device 500 may be configured to deliver the nicotine or THC solution 112 similar to the device 100a or inhalation unit 110b-c. The device 500 may also include one or more user input devices 510 and 520 configured to adjust the predetermined maximum nicotine or THC consumption or user nicotine, cotinine, or THC level. The user input devices 510 and 520 may be, for example, dials or switches connected to a variable resistor. The inhalation sensor 200 may adjust the predetermined maximum nicotine or THC consumption or user nicotine, cotinine, or THC level based on the location of the user input devices 510 and 520.

In the exemplary embodiment illustrated in FIG. 5, the user input device 510 may be used to adjust the predetermined maximum nicotine/THC consumption for one session while the user input device 520 may be used to adjust a the predetermined maximum nicotine/THC consumption for one rolling 24 hour period. The user input devices 510 and 520 and inhalation sensor 200 may be calibrated such that user input devices 510 and 520 are aligned with visual indicators such as hash marks 512 and 522. The number of user input devices and the degree of freedom available for each user input device may be constrained by the size of the device 500. Alternatively, the predetermined maximum nicotine/THC consumption or user nicotine/cotinine/THC levels may be preprogrammed or adjustable by a user through an external device in communication with the device 500 wireless or wired connection as described above.

Alternatively, the device 500 may include user input devices similar to user input devices 510 and 520 that allow a user to input one or more user biometrics. For example, the device may include a user input device that allows a user to input the user's weight.

The alternative nicotine/THC delivery device may include one or more alternate reservoirs that may include a reduced concentration of nicotine/THC solution 112 (either in addition to the reservoir 110 or in a separate portion of the reservoir 110). The alternate reservoir may enable a user to manually switch to a reduced concentration of nicotine/THC solution. Alternatively, the alternative nicotine/THC delivery device may automatically vaporize the reduced concentration nicotine/THC solution 112 in response to a determination by the inhalation sensor 200 that the user has consumed a predetermined maximum nicotine or THC amount or the user nicotine/cotinine/THC levels has reached a predetermined maximum level.

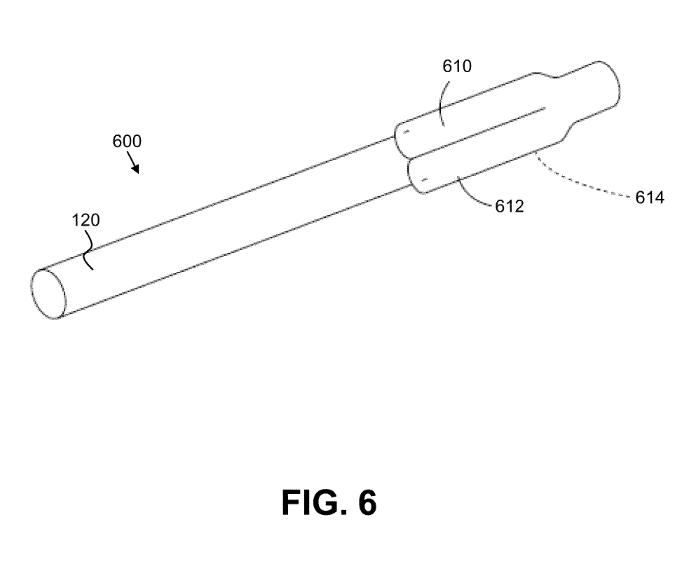

FIG. 6 illustrates an alternative nicotine/THC delivery device 600 according to another exemplary embodiment of the present invention. The device 600 may be configured to deliver a nicotine/THC solution 112 similar to the device 100a or inhalation unit 110b-c. The device 600 may also include reservoirs 610, 612, and 614, for containing solution with three distinct concentrations of nicotine or THC.

For example, the reservoir 610 may include the highest concentration of nicotine or THC, reservoir 612 may include a reduced-concentration solution, and the reservoir 614 (on the back side of the device 600 relative to the viewer) may include a solution with no nicotine or THC. The device 600 may be configured to vaporize the solution in reservoir 612 until the user has reached or exceeded the predetermined nicotine or THC consumption target in a given time period, and then vaporize the reduced-concentrated solution (for example, by an alternate heating element and/or a reservoir injection system) in reservoir 614 until nicotine or THC consumption levels are reduced below the target level (or another pre-defined level of nicotine or THC consumption). The device 600 may also be configured to vaporize the solution in reservoir 614 if the user has reached or exceeded a second predetermined nicotine/THC consumption target in a given time period.

Alternatively, the device 600 may include an on-demand solution mixing device for electromechanically mixing pure nicotine or THC and solvents in a reservoir or directly into the heating element in order to provide a reduced-concentration solution 112.

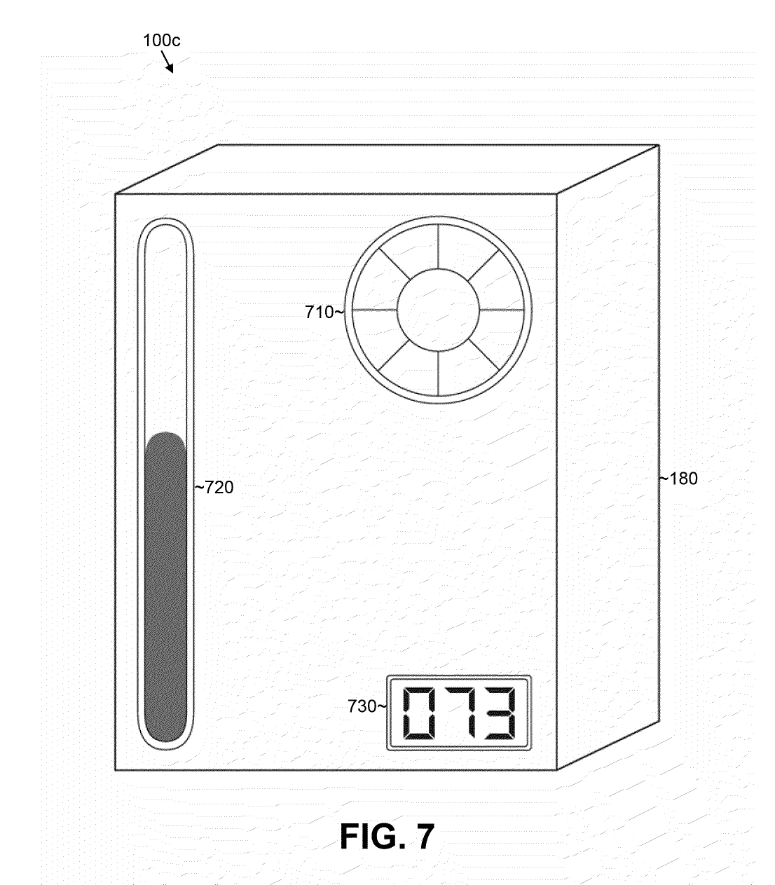

FIG. 7 illustrates another view of the charging unit 180a illustrated in FIG. 1B and/or the charging unit 180b illustrated in FIG. 1C. As illustrated in FIG. 7, the charging unit 180 may include an input/output device 710, a visual indicator 720, and/or a visual indicator 730.

The input/output device 710 may any suitable device configured to receive input from a user and/or display information to a user. The input/output device 710 may be configured to enable a user to input or adjust the predetermined maximum nicotine/THC consumption level, the predetermined maximum user nicotine/cotinine/THC level, static or dynamic biometric information, etc. The input/output device 710 may also be configured to output (e.g., via a display) the predetermined maximum nicotine/THC consumption level, the maximum user nicotine/cotinine/THC level, static or dynamic biometric information, etc.

The visual indicator 720 or 730 may output information indicative of the user nicotine/THC consumption or user nicotine/cotinine/THC levels. Similar to the visual indicators 310, 320, 330, and 410, the visual indicator 720 or 730 may be any suitable device configured to selectively emit or reflect light.

In each of the exemplary embodiments described above, the inhalation sensor 200 may be used as a smoking cessation device. In some instances, the simple act of informing the user regarding nicotine or THC consumption or user nicotine, cotinine, or THC levels may reduce nicotine or THC consumption. In other instances, the user may be restricted from exceeding a predetermined maximum nicotine or THC consumption or user nicotine, cotinine, or THC level.

The inhalation sensor 200 may be configured to reduce the predetermined maximum nicotine/THC consumption or user nicotine/cotinine/THC level over a large time window (for example, days, weeks, months, etc.). The inhalation sensor 200 may be configured to account for human models of withdrawal. For instance, the inhalation sensor 200 may allow for high impulses upon wake and then further tapering throughout wakeful hours. Alternatively, the inhalation sensor 200 may be configured to mimic traditional cigarette profiles. For example, a user may prefer a nicotine consumption limit equivalent to one pack of traditional cigarettes per day, spaced out into 20 equal doses equivalent to one traditional cigarette each.

While exemplary embodiments have been set forth above, those skilled in the art who have reviewed the present disclosure will readily appreciate that other embodiments can be realized within the scope of the invention. For example, while this disclosure describes regulating five distinct measures of nicotine or THC consumption with respect to time (peak nicotine/THC plasma levels, average nicotine/THC plasma levels, cumulative nicotine/THC consumption over a sliding window, cotinine levels over a sliding window, and nicotine/THC consumption per session) it is to be understood that other metrics are simply reconfiguration of the same logic (programmable variants) and should be encompassed by this application. Therefore, the present invention should be construed as limited only by the appended claims.

What is claimed is:

1. An inhalation sensor for an alternative nicotine or THC delivery device including a diluted nicotine or THC solution, the inhalation sensor comprising:
a processor; and
memory comprising:
a solution profile including information indicative of the nicotine or THC concentration of the diluted nicotine or THC solution; and
a device profile including information indicative of the nicotine or THC delivery device,
wherein the inhalation sensor is configured to determine an amount of nicotine or THC consumed by a user of the alternative nicotine or THC delivery device based on the solution profile and the device profile.

2. The inhalation sensor of claim 1, further comprising a timer and an inhalation timing sensor configured to detect an inhalation by a user,
wherein the inhalation sensor is configured to determine the amount of nicotine or THC consumed by the user further based on a duration of the inhalation.

3. The inhalation sensor of claim 2, wherein the inhalation sensor is configured to determine the amount of nicotine or THC consumed by the user further based on information indicative of a temperature of a heating element configured to vaporize the diluted nicotine or THC solution.

4. The inhalation sensor of claim 3, wherein the inhalation sensor is configured to determine the information indicative of the temperature of the heating element based on the duration of the inhalation.

5. The inhalation sensor of claim 4, wherein the device profile includes information indicative of a relationship between the duration of the inhalation and the temperature of the heating element.

6. The inhalation sensor of claim 3, wherein the inhalation sensor is configured to determine the amount of nicotine or THC consumed by the user further based on a time between the inhalation and a previous inhalation.

7. The inhalation sensor of claim 6, wherein the device profile further includes a ramp up profile indicative of the temperature of the heating element during inhalation and a decaying profile indicative of the temperature of the heating element after the heating element is de-activated.

8. The inhalation sensor of claim 3, wherein the solution profile further includes a burning point of the nicotine or THC solution.

9. The inhalation sensor of claim 8, wherein the inhalation sensor is further configured to determine a temperature of the heating element and compare the temperature of the heating element to the boiling point of the nicotine or THC solution.

10. The inhalation sensor of claim 9, wherein the inhalation sensor is configured to output an indication to the user in response to the temperature of the heating element approaching or exceeding the boiling point of the nicotine or THC solution.

11. The inhalation sensor of claim 9, wherein the inhalation sensor is configured to output a signal to the alternative nicotine or THC delivery device to interrupt the supply of power to the heating element in response to a determination that the temperature of the heating element approaching or exceeding the boiling point of the nicotine or THC solution.

12. The inhalation sensor of claim 1, further comprising:
a visual indicator configured to output a visual indication indicative of the amount of nicotine or THC consumed by the user.

13. The inhalation sensor of claim 12, wherein the visual indication is indicative of a comparison of the amount of nicotine or THC consumed by the user and an amount of nicotine or THC in a traditional cigarette.

14. The inhalation sensor of claim 1, wherein the inhalation sensor is configured to compare the amount of nicotine or THC consumed by the user to a nicotine or THC consumption limit.

15. The inhalation sensor of claim 14, wherein the nicotine or THC consumption limit is user adjustable.

16. The inhalation sensor of claim 15, wherein the inhalation sensor is configured to reduce the nicotine or THC consumption limit over time.

17. The inhalation sensor of claim 14, wherein the inhalation sensor is configured to output a signal to the alternative nicotine or THC delivery device to prevent further delivery of the diluted nicotine or THC solution in response to a determination that the amount of nicotine or THC consumed is greater than or equal to the nicotine or THC consumption limit.

18. The inhalation sensor of claim 14, wherein the inhalation sensor is configured to output a signal to the alternative nicotine or THC delivery device to interrupt a supply of power to a heating element in response to a determination that the amount of nicotine or THC consumed is greater than or equal to the nicotine or THC consumption limit.

19. The inhalation sensor of claim 14, wherein the inhalation sensor is configured to output a signal to the alternative nicotine or THC delivery device to interrupt a flow of the diluted nicotine or THC solution in response to a determination that the amount of nicotine or THC consumed is greater than or equal to the nicotine or THC consumption limit.

20. The inhalation sensor of claim 1, wherein the inhalation sensor is configured to determine the amount of nicotine or THC consumed by the user further based on biometrics of the user.
